(19) United States
(12) Patent Application Publication
(10) Pub. No.: US 2016/0354402 A1
(43) Pub. Date: Dec. 8, 2016

(54) COMPOSITIONS

(71) Applicant: GLOBALACORN LTD, London (GB)

(72) Inventors: Andrew David Miller, London (GB); Natalya Lozovaya, Marseille (FR); Nail Burnashev, Marseille Amerag (FR); Rashid Giniatullin, (US)

(21) Appl. No.: 15/039,750

(22) PCT Filed: Nov. 27, 2014
(86) PCT No.: PCT/GB2014/053522

§ 371 Date: May 26, 2016

(30) Foreign Application Priority Data
Nov. 27, 2013 (GB) 1320959.8

(51) Int. Cl. A61K 31/7084

(52) U.S. Cl. CPC A61K 31/7084

(57) ABSTRACT

The invention provides a dinucleoside polyphosphate analogue, or a pharmaceutically acceptable salt thereof, for use as an anticonvulsant and/or seizure suppressant, in particular in the treatment or prevention of (e.g. juvenile) epilepsy.

### FIELD OF THE INVENTION

The present invention relates to the use of (analogues of) dinucleoside polyphosphates and other compounds as an anticonvulsant and/or seizure suppressant, more particularly for the treatment (or prevention, suppression and/or reduction) of epilepsy, and so act as an anti-epileptic agent.

### BACKGROUND TO THE INVENTION

Epilepsy is a common and diverse set of chronic neurological disorders characterized by seizures. Epileptic seizures result from abnormal, excessive or hypersynchronous neuronal activity in the brain. About 50 million people worldwide have epilepsy, and nearly 80% of epilepsy occurs in developing countries. Epilepsy becomes more common as people age.

Epilepsy is usually controlled, but not cured, with medication. However, more than 30% of people with epilepsy do not have seizure control even with the best available medications. In addition, different epileptic syndromes may respond to different medications, and not all epileptic syndromes are susceptible to pharmacological control.

### SUMMARY OF THE INVENTION

The present invention represents can alleviate (some of) the problems of the prior art.

In one aspect, the present invention provides a dinucleoside polyphosphate (analogue), or a pharmaceutically acceptable salt thereof, for use as an anticonvulsant and/or seizure suppressant, more particularly for the treatment (or prevention or reduction) of epilepsy. Thus, the present invention also provides a dinucleoside polyphosphate (analogue), or a pharmaceutically acceptable salt thereof, for use in the treatment of epilepsy.

In another aspect, the present invention provides a method of treatment, suppression or prevention of convulsions and/or seizures, comprising administering an effective amount of a dinucleoside polyphosphate polyphosphate (analogue) or a pharmaceutically acceptable salt thereof.

The invention further provides the use of a dinucleoside polyphosphate (analogue) or a pharmaceutically acceptable salt thereof in the manufacture of a medicament for the treatment, suppression or prevention of convulsions and/or seizures.

### BRIEF DESCRIPTION OF THE DRAWINGS

FIG. 1 Animal model of epilepsy spontaneous seizures in Tsc1^{+/-} mice. (a) EEG recorded Intracortical in a head-restrained P16 Tsc1^{+/-} mouse at 8 layer positions (L1-8) using a 16 channels silicone probe. The upper channel corresponds to the superficial intracortical electrode placed at the uppermost layer (L1) (100 μm from the pia). Also shown are epileptic discharges recorded in different layers (L2-8) at increasing depths indicated on the left of each trace. (b) Superimposed epileptic discharges in layer L4 in neocortex (red) and in hippocampus (black). (c) Wavelet analysis during the ictal events in layer L4 traces (shown in b), with upper panel: neocortex, lower panel: hippocampus. (d) Cumulative probabilities of seizures maximal amplitudes seen in layers L2/3 and L4 (upper left) and durations (upper right). Seizure durations were the same at all layers, data for layers L2/3 are shown. Bottom: Relative integral power of δ-(1-4 Hz) θ-(4-8 Hz), α-(8-12), β-(12-25 Hz), γ-(25-100 Hz) and fast ripple (FR, 100-500 Hz) band components of EEG in L2/3 and L4 revealed by Fourier transform analysis.

FIG. 2 Spontaneous seizures in Tsc1^{+/-} mice. (a) Experimental setup for the 16-channel silicone probe detection of spontaneous seizures recorded in different layers (L1-6) of the somatosensory cortex of P15 Tsc1^{+/-} mouse. CUX-1 staining is used to identify layers L1-L4 cortical layers (left panel). (b) Example of intracortical EEG recordings (2 h) in a head-restrained P15 Tsc1^{+/-} mouse without any pharmacological treatment. The upper trace corresponds to the uppermost cortical layer (L1) with electrode placed at 100 μm (from the pia). Epileptic discharges are recorded in most layers of different depths (indicated on the left of each trace) but at different times.

FIG. 3 Acute antiepileptic effect of AppCH₂ppA (100 μM) in vivo post i.p.-administration. (a) Intracortical EEG recordings in head-restrained P15 Tsc1^{+/-} mouse before and after i.p.-administration of AppCH₂ppA (at position indicated by arrow). The upper trace corresponds to the superficial intracortical electrode placed in the uppermost cortical layer (L1) (100 μm from the pia), other traces were recorded in layers (L2-5) separated by 200 μm. (b) Time course of spontaneous seizure activity in Tsc1^{+/-} mice at P14-P16 before and after i.p.-administration of AppCH₂ppA (lower panel) and vehicle control (upper panel). Individual seizures are represented by black squares. Each row represents individual experiments. Administration of the dinucleoside polyphosphate eliminates seizures virtually completely.

FIG. 4 Acute antiepileptic effect of AppCH₂ppA (30 μM) in vivo post i.p.-administration. (a) Intracortical EEG recordings in head-restrained P15 Tsc1^{+/-} mouse before and after i.p.-administration of AppCH₂ppA (30 μM) (indicated by arrow). The upper trace corresponds to the superficial intracortical electrode placed in the uppermost cortical layer (L1) (100 m from the pia), other traces were recorded in layers (L1-5) separated by 200 m.

FIG. 5A Antiepileptic effect of AppCH₂ppA (10 μM) ex vivo post administration to cortical slices from Tsc1+/− mice (A). Whole-cell patch-clamp recordings are shown of spontaneous glutamatergic activity from L5 interneurons under control conditions and after bath administration of AppCH₂ppA (Vh=−70 mV) (10 M); ); (B) Top 3 panels (left to right) are control, AppCH₂ppA treated, and washout: bottom panel demonstrates that AppCH₂ppA desensitizes glutamatergic activity relative to control and thereby reduces the likelihood and/or frequency of epileptic discharges.

FIG. 6 Summary of the proposed mechanism for the antiepileptic effect of AppCH₂ppA

FIG. 7 Epilepsy model established ex vivo in mouse hippocampal slices. Current-(upper panels) and voltage-(lower panels) clamp recordings from CA1 pyramidal neurons in hippocampal slices in normal (a) and epileptic conditions (b). Epileptic conditions were established through the addition of picrotoxin (100 μM) and removal of Mg²⁺ in the slice perfusion solution.

FIG. 8 Antiepileptic effect of AppCH₂ppA ex vivo in mouse hippocampal slices. Current (a) and voltage-(b) clamp recordings from CA1 pyramidal neurons in hippocampal slices in epileptic conditions before, during and post administration of AppCH₂ppA (10 M). Panel shows giant (epileptiform) spontaneous excitatory postsynaptic currents (EPSCs) superimposed in absence (1) and in the presence of AppCH₂ppA (2) (10 M).

FIG. 9 Antiepileptic effects of selected dinucleoside polyphosphate analogues ex vivo in mouse hippocampal slices. (a) AppCH₂ppA dose response effects on frequency of epileptiform discharges in epileptic conditions; (b) AppNHppA dose response effects on frequency of epileptiform discharges in epileptic conditions; (c) representative trace of the current-clamp recordings from the hippocampal CA1 pyramidal neurons in epileptic conditions in the presence of AppNHppA at the indicated concentrations; (d) dose response effects on frequency of epileptiform discharges in epileptic conditions post administration of indicated dinucleoside polyphosphate analogues.

### DETAILED DESCRIPTION OF THE INVENTION

The invention uses dinucleoside polyphosphates, a family of compounds comprising two nucleoside moieties linked by a polyphosphate bridge. They can be represented by NpₙN, wherein N represents a nucleoside moiety, p represents a phosphate group and n is the number of phosphate groups (e.g. 2 to 7). Analogues of dinucleoside polyphosphates are compounds (typically synthetic) having a structure based on that of a dinucleoside polyphosphate, wherein one or more parts of the structure have been altered. For example the nucleobase, the sugar and/or the phosphate backbone may be modified, or partially or fully replaced, by another suitable moiety.

For example, one or more polyphosphate chain oxo-bridges may be replaced by a different bridge to increase the biological half-life of the compound in vivo. Such analogues may be designed to provide stability and/or biocompatibility. To achieve this, the analogue should be resistant to decomposition by biological systems in vivo. For example, the analogue may have increased hydrolytic stability, i.e. resistance to the breakdown of the molecule by specific enzyme cleavage (e.g. by one or more types of nucleotidase) and/or non-specific hydrolysis.

Preferably the compounds are diadenosine polyphosphates (e.g. of the type ApₙAs; where n is 2-7), such as naturally occurring purinergic ligands consisting of two adenosine moieties bridged by a chain of two or more phosphate residues attached at the 5′-position of each ribose ring. In particular, P¹, P⁴-diadenosine tetraphosphate (Ap₄A) and P¹, P⁵-diadenosine pentaphosphate (Ap₅A) are contemplated. These are present in high concentrations endogenously in the secretory granules of chromaffin cells and in rat brain synaptic terminals. Upon depolarization, ApₙAs are released in a Ca²⁺-dependent manner and their potential role as neurotransmitters has been proposed. However, in spite of being well known for many years, pure functions of ApₙAs have been difficult to define because of both specific enzymatic cleavage and nonspecific hydrolytic breakdown. ApₙA analogues can be more stable than naturally occurring diadenosine polyphosphates with respect to both specific enzymatic and nonspecific hydrolytic breakdown.

### Preferred Compounds

Preferably, the dinucleoside polyphosphate (of the NPₙ N type) for use in the present invention is a compound of formula (I):

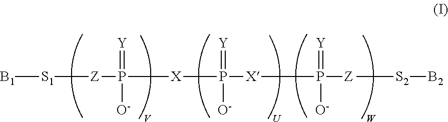

or a pharmaceutically acceptable salt thereof,
wherein X, X′ and Z are independently selected from

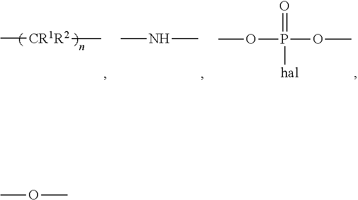

wherein R¹ and R² are independently selected from hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl, and n is selected from 1, 2, 3, 4, 5 and 6;
each Y is independently selected from ═S and ═O;
B₁ and B₂ are independently selected from a 5- to 7-membered carbon-nitrogen heteroaryl group which may be unfused or fused to a further 5- to 7-membered carbon-nitrogen heteroaryl group
S₁ and S₂ are independently selected from a bond, C₁₋₆ alkylene, C₂₋₆ alkenylene, C₂₋₆ alkynylene and a moiety of formula (II):

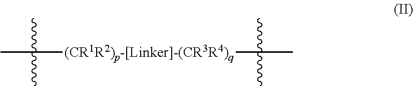

wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0, 1, 2 or 3, preferably 0, 1 or 2; and
[Linker] represents:
   (i) —O—, —S—, —C═O— or —NH—;
   (ii) C₁₋₄ alkylene, C₂₋₄ alkenylene or C₂₋₄ alkynylene, which may optionally contain or terminate in an ether (—O—), thioether (—S—), carbonyl (—C═O—) or amino (—NH—) link, and which are optionally substituted with one or more groups selected from hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; or
   (iii) a 5 to 7 membered heterocyclyl, carbocyclyl or aryl group, which may be optionally substituted with one or more groups selected from hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl;
      V is selected from 0, 1, 2, 3, 4 and 5;
      U is selected from 0, 1, 2, 3, 4 and 5;
      W is selected from 0, 1, 2, 3, 4 and 5; and
      V plus U plus W is an integer from 2 to 7.

As used herein, a C₁₋₄ alkyl group or moiety is a linear or branched alkyl group or moiety containing from 1 to 4 carbon atoms. Examples of C₁₋₄ alkyl groups include methyl, ethyl, n-propyl, i-propyl, n-butyl, i-butyl and t-butyl.

As used herein, a C₂₋₄ alkenyl group or moiety is a linear or branched alkenyl group or moiety having at least one double bond of either E or Z stereochemistry where applicable and containing from 2 to 4 carbon atoms, such as —CH═CH₂ or —CH₂—CH═CH₂, —CH₂—CH₂—CH═CH₂, —CH₂—CH═CH—CH₃, —CH═C(CH₃)—CH₃ and —CH₂—C(CH₃)═CH₂.

As used herein, a C₁₋₆ alkylene group or moiety is a linear or branched alkylene group or moiety, for example a C₁₋₄ alkylene group or moiety. Examples include methylene, n-ethylene, n-propylene and —C(CH₃)₂— groups and moieties.

As used herein, a C₂₋₆ alkenylene group or moiety is a linear or branched alkenylene group or moiety, for example a C₂₋₄ alkenylene group or moiety. Examples include —CH═CH—, —CH═CH—CH₂—, —CH₂—CH═CH— and —CH═CH—CH═CH—.

As used herein, a C₂₋₆ alkynylene group or moiety is a linear or branched alkynylene group or moiety, for example a C₂₋₄ alkynylene group or moiety. Examples include —C≡C—, —C≡C—CH₂— and —CH₂—C≡C—.

As used herein, a halogen atom is chlorine, fluorine, bromine or iodine.

As used herein, a C₁₋₄ alkoxy group or C₂₋₄ alkenyloxy group is typically a said C₁₋₄ alkyl group or a said C₂₋₄ alkenyl group respectively which is attached to an oxygen atom.

A haloalkyl or haloalkenyl group is typically a said alkyl or alkenyl group respectively which is substituted by one or more said halogen atoms. Typically, it is substituted by 1, 2 or 3 said halogen atoms. Preferred haloalkyl groups include perhaloalkyl groups such as —CX₃ wherein X is a said halogen atom, for example chlorine or fluorine.

Preferably, a C₁₋₄ or C₁₋₃ haloalkyl group as used herein is a C₁₋₃ fluoroalkyl or C₁₋₃ chloroalkyl group, more preferably a C₁₋₃ fluoroalkyl group.

As used herein, a C₁₋₄ aminoalkyl group is a C₁₋₄ alkyl group substituted by one or more amino groups. Typically, it is substituted by one, two or three amino groups. Preferably, it is substituted by a single amino group.

As used herein, a C₁₋₄ hydroxyalkyl group is a C₁₋₄ alkyl group substituted by one or more hydroxy groups. Typically, it is substituted by one, two or three hydroxy groups. Preferably, it is substituted by a single hydroxy group.

As used herein, a C₁₋₄ acyl group is a group —C(═O)R, wherein R is a said C₁₋₄ alkyl group.

As used herein, a 5 to 7 membered heterocyclyl group includes heteroaryl groups, and in its non-aromatic meaning relates to a saturated or unsaturated non-aromatic moiety having 5, 6 or 7 ring atoms and containing one or more, for example 1 or 2, heteroatoms selected from S, N and O, preferably O. Illustrative of such moieties are tetrahydrofuranyl and tetrahydropyranyl. For example, the heterocyclic ring may be a furanose or pyranose ring.

As used herein, a 5- to 7-membered carbon-nitrogen heteroaryl group is a monocyclic 5- to 7-membered aromatic ring, such as a 5- or 6-membered ring, containing at least one nitrogen atom, for example 1, 2, 3 or 4 nitrogen atoms. The 5- to 7-membered carbon-nitrogen heteroaryl group may be fused to another 5- to 7-membered carbon-nitrogen heteroaryl group.

As used herein, a 5 to 7 membered carbocyclyl group is a non-aromatic, saturated or unsaturated hydrocarbon ring having from 5 to 7 carbon atoms. Preferably it is a saturated or mono-unsaturated hydrocarbon ring (i.e. a cycloalkyl moiety or a cycloalkenyl moiety) having from 5 to 7 carbon atoms. Examples include cyclopentyl, cyclohexyl, cyclopentenyl and cyclohexenyl.

As used herein, a 5 to 7 membered aryl group is a monocyclic, 5- to 7-membered aromatic hydrocarbon ring having from 5 to 7 carbon atoms, for example phenyl.

In one aspect X and X′ are independently —NH—. However, in some compounds neither X or X′ are —NH—.

In one aspect X and X′ are independently

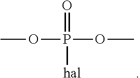

In one aspect X and X′ are independently

CR¹R²ₙ,

wherein at least one of R¹ and R² is H, Cl, Br or F.

Preferably both R¹ and R² are H.

Preferably n is 1, 2 or 3, preferably 1 or 2.

Preferably at least one of X and X′ is not —O—, i.e. not all X and X′ are —O—.

Preferably X and X′ are independently selected from NH and

CR¹R²ₙ

wherein R¹ and R² are both H and n is 1 or 2.

In one aspect at least one Y is ═S.

In one aspect each Y group is ═S.

In one aspect at least one Y is ═O.

Preferably each Y group is ═O.

In one aspect at least one Z is

CR¹R²ₙ.

In one aspect each Z is

CR¹R²ₙ

wherein at least one of R¹ and R² is H, Cl, Br or F.

Preferably both R¹ and R² are H. Thus, in one aspect Z is

CR¹R²ₙ

and R¹ and R² are both H.

Preferably n is 1, 2 or 3, preferably 1 or 2.

In one aspect at least one Z is —NH—.

In one aspect each Z is —NH—.

In one aspect at least one Z is —O—.

Preferably each Z is —O—.

B₁ and B₂ are preferably independently selected from purine and pyrimidine nucleic acid bases, preferably adenine, guanine, thymine, cytosine, uracil, hypoxanthine, xanthine, 1-methyladenine, 7-methylguanine, 2-N,N-dimethylguanine, 5-methylcytosine or 5,6-dihydrouracil. Uracil may be attached to S or S₂ via N (i.e. uridine structure) or C (i.e. pseudouridine structure).

Preferably, B₁ and B₂ are independently selected from adenine, guanine, and uracil.

Preferably at least one of B₁ and B₂ is adenine.

Thus, for example, at least one of B₁ and B₂ may be adenine and the other of B₁ and B₂ may be guanine, or at least one of B₁ and B₂ may be adenine and the other of B₁ and B₂ may be uracil.

S₁ and S₂ are preferably independently selected from a bond, C₁₋₆ alkylene, C₂₋₆ alkenylene, C₂₋₆ alkynylene and a moiety of formula (III) or (IV):

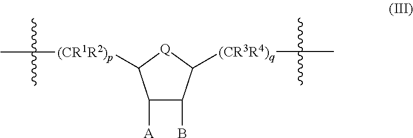

wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0 or 1;
Q represents —O—, —S—, —C═O—, —NH— or CH₂; and
A and B independently represent hydrogen, hydroxyl, halogen, or an unsubstituted group selected from C₁₋₄ alkoxy, C₁₋₄ aminoalkyl, C₁₋₄ hydroxyalkyl, C₁₋₄ acyl and —NR⁵R⁶ groups, wherein R⁵ and R⁶ are the same or different and represent hydrogen or unsubstituted C₁₋₂ alkyl;

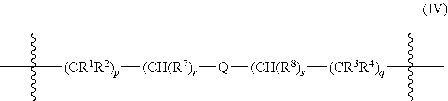

wherein
R¹, R², R³ and R⁴ independently represent hydrogen, halogen, cyano or an unsubstituted group selected from C₁₋₃ haloalkyl, C₁₋₃ alkyl, C₁₋₄ aminoalkyl and C₁₋₄ hydroxyalkyl;
Q represents —O—, —S—, —C═O—, —NH— or CH₂; and
R⁷ and R⁸ independently represent hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; and
p, q, r and s independently represent 0 or 1.

S₁ and S₂ are preferably independently selected from a moiety of formula (III) or (IV) as set out above, in which preferably:
R¹, R², R³ and R⁴ independently represent hydrogen, fluoro, chloro, or unsubstituted C₁₋₃ alkyl; more preferably hydrogen;
Q represents —O—;
A and B independently represent hydrogen, hydroxyl, fluoro, chloro, methoxy, formyl or NH₂, more preferably hydrogen or hydroxyl; and
R⁷ and R⁸ independently represent hydrogen, hydroxyl, fluoro, chloro, or an unsubstituted group selected from C₁₋₄ alkyl, C₁₋₄ haloalkyl, C₁₋₄ hydroxyalkyl and C₁₋₄ alkyl-NH₂, more preferably hydrogen, hydroxyl or unsubstituted methyl, ethyl, —CH₂OH or —CH₂CH₂OH.

S₁ and S₂ may preferably be independently selected from D-ribofuranose, 2′-deoxy-D-ribofuranose, 3′-deoxy-D-ribofuranose, L-arabinofuranose (corresponding to moieties of formula (III)), and ring opened forms thereof (corresponding to moieties of formula (IV)).

In one preferred embodiment, at least one of S₁ and S₂ is D-ribofuranose, i.e. a moiety of formula (III′) in which R¹ and R² are hydrogen, p is 1, q is 0, Q is —O— and A and B are hydroxyl:

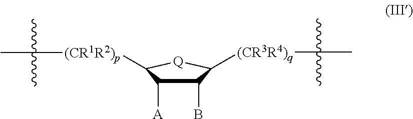

When S₁ and/or S₂ is a ring opened form, the ring opening is preferably between the 2′ and 3′ positions of the D-ribofuranose, 2′-deoxy-D-ribofuranose, 3′-deoxy-D-ribofuranose or L-arabinofuranose ring.

In one preferred embodiment, at least one of S₁ and S₂ is a ring opened form of D-ribofuranose, for example a moiety of formula (IV) in which R¹ and R² are hydrogen, p is 1, q is 0, Q is —O—, r is 1, s is 1 and R⁷ and R⁸ are each —CH₂OH.

Preferably S₁ and S₂ are the same. Thus preferably, S₁ and S₂ are both D-ribofuranose or both a ring opened form of D-ribofuranose as described above.

The sum of V, U and W may be 2, 3, 4, 5, 6 or 7.

Preferably V plus U plus W is 4 or 5.

Preferably U is 0, 1 or 2.

Preferably V is 2.

Preferably W is 2.

In a preferred embodiment, U is 0. Thus the dinucleoside polyphosphate for use in the present invention is preferably a compound of formula (I′):

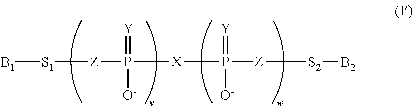

wherein all symbols are as defined above, X is not —O— and V plus W is a integer from 2 to 7.

Thus, the sum of V and W in formula (I′) may be 2, 3, 4, 5, 6 or 7. Preferably V plus W is 4 or 5. Preferably V is 2 and/or W is 2 or 3.

In a preferred embodiment, each Y is ═O and each Z is —O—. In some compounds X is not —NH—.

In a more preferred embodiment, each Y is ═O and each Z is —O—, and both S₁ and S₂ are a moiety of formula (III) or (IV) as set out above. Preferably, both S₁ and S₂ are the same and are both D-ribofuranose or both a ring opened form of D-ribofuranose. Thus the dinucleoside polyphosphate analogue of the present invention is preferably a compound of formula (IA) or (IB):

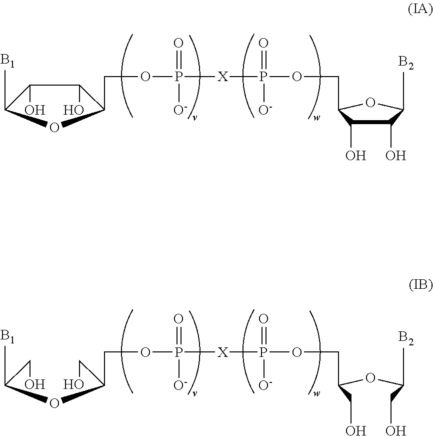

Preferably, the dinucleoside polyphosphate analogue of the present invention is a compound of formula (IA) or (IB) wherein V plus W is 4 or 5. More preferably, the dinucleoside polyphosphate analogue of the present invention is a compound of formula (IA) or (IB) wherein at least one of B₁ and B₂ is adenine, or one of B₁ and B₂ is adenine and the other is guanine.

Thus, in a more preferred embodiment, each Y is ═O and each Z is —O—, both S₁ and S₂ are the same and are both D-ribofuranose or both a ring opened form of D-ribofuranose, and B₁ and B₂ are both adenine, or one of B₁ and B₂ is adenine and the other is guanine or uracil. Thus the dinucleoside polyphosphate analogue of the present invention may preferably be a dinucleoside polyphosphate compound of formula (IC) to (IH):

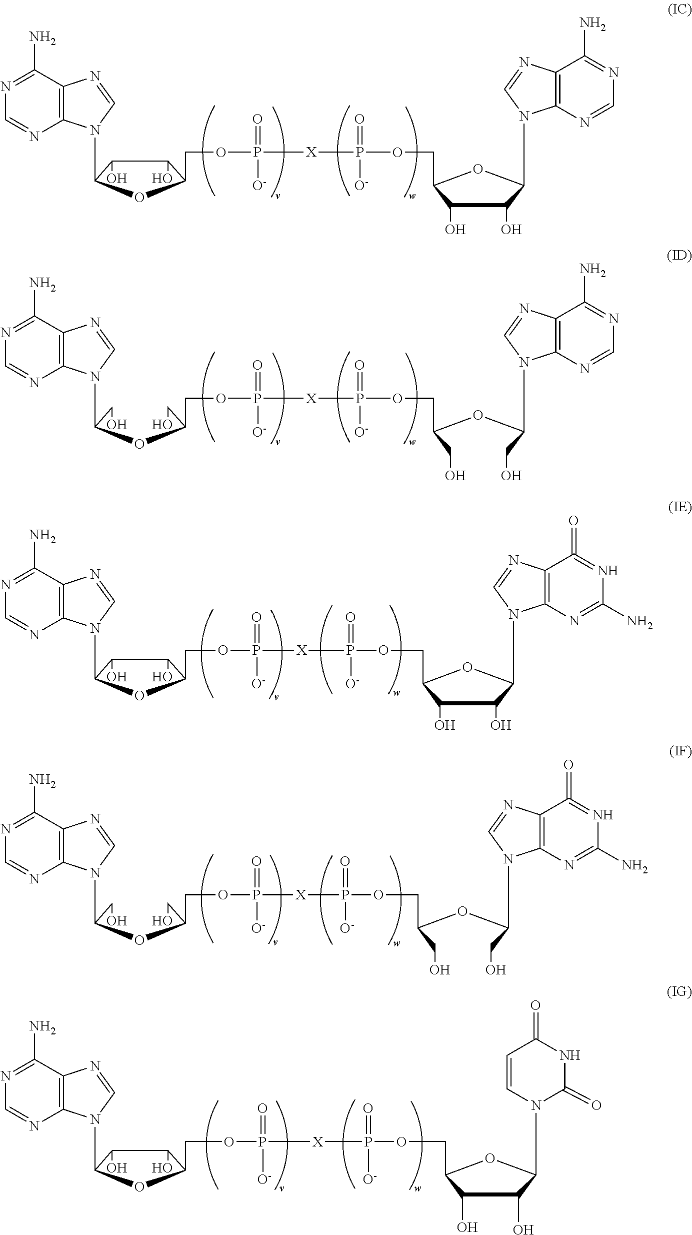

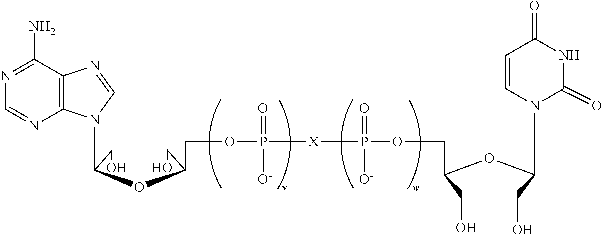

Preferably, the dinucleoside polyphosphate analogue is a compound of formula (IC) to (IH) wherein V plus W is 4 or 5. Thus, in a preferred aspect of the invention, the dinucleoside polyphosphate analogue is chosen among the group consisting of Ap₄A analogues, Ap₅A analogues, Ap₄G analogues, Ap₅G analogues, Ap₄U analogues and Ap₅U analogues.

In one embodiment, V and W are the same. Thus in the above compounds of formula (I′) and (IA) to (IH), V and W may each be 2. In a further embodiment, the dinucleoside polyphosphate analogue may be symmetrical.

In a preferred aspect of the invention, the dinucleoside polyphosphate analogue is chosen among the group consisting of AppCH₂ppA, AppNHpppU, A_{diol}ppCH₂ppA_{diol}, A_{diol}ppNHppA_{diol}, AppCH₂ppG, AppNHppG, A_{diol}ppCH₂ppG_{diol} and A_{diol}ppNHppG_{diol}:

AppCH₂ppA:

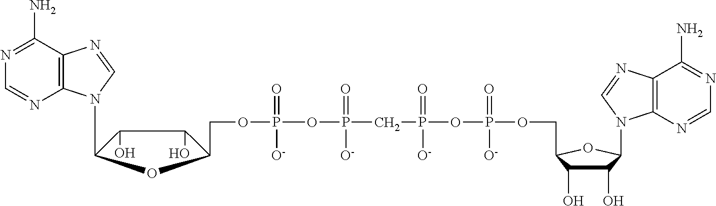

AppNHpppU:

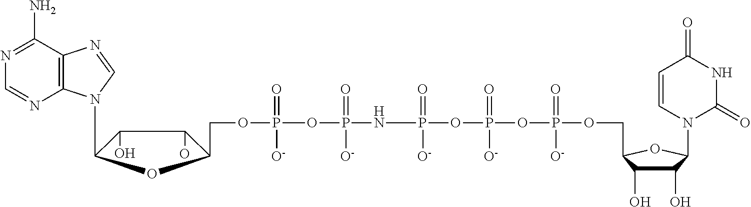

A_{diol}ppCH₂ppA_{diol}:

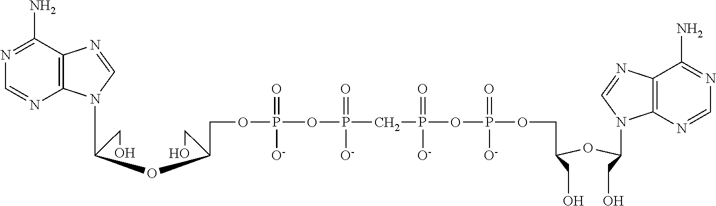 [SMILES: Nc1ncnc2c1ncn2[C@@H](CO)O[C@@H](CO)COP(=O)([O-])OP(=O)([O-])CP(=O)([O-])OP(=O)([O-])OC[C@H](CO)O[C@H](CO)n1cnc2c(N)ncnc21]

A_{diol}ppNHppA_{diol}:

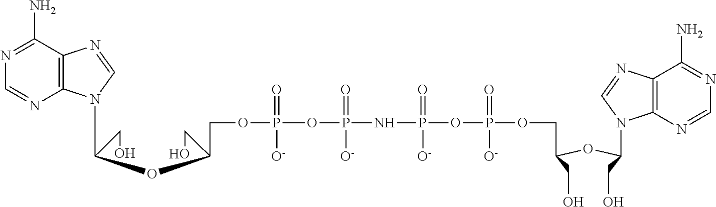 [SMILES: Nc1ncnc2c1ncn2[C@@H](CO)O[C@@H](CO)COP(=O)([O-])OP(=O)([O-])NP(=O)([O-])OP(=O)([O-])OC[C@H](CO)O[C@H](CO)n1cnc2c(N)ncnc21]

AppCH₂ppG:

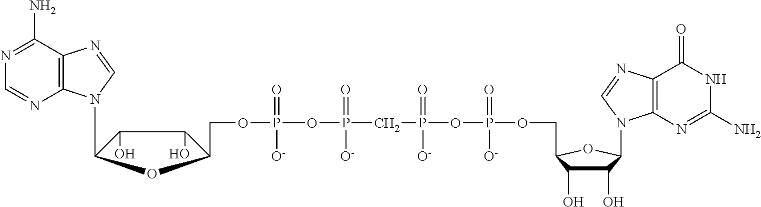

AppNHppG:

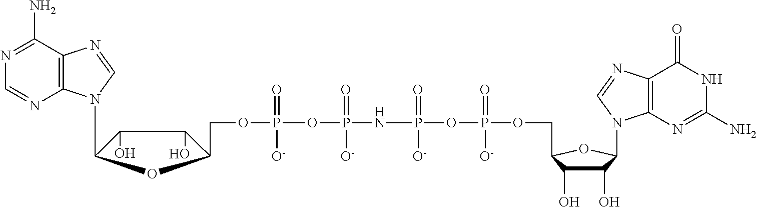

A_{diol}ppCH₂ppG_{diol}:

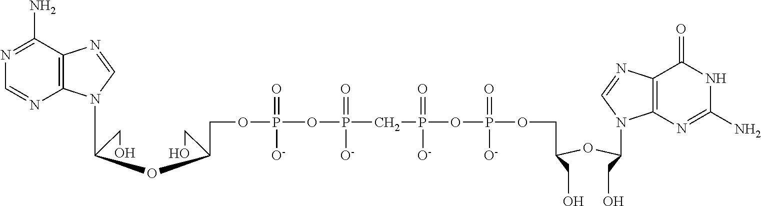 [SMILES: Nc1nc2c(ncn2[C@@H](CO)O[C@@H](CO)COP(=O)([O-])OP(=O)([O-])CP(=O)([O-])OP(=O)([O-])OC[C@H](CO)O[C@H](CO)n2cnc3c(N)ncnc32)c(=O)[nH]1]

A_{diol}ppNHppG_{diol}:

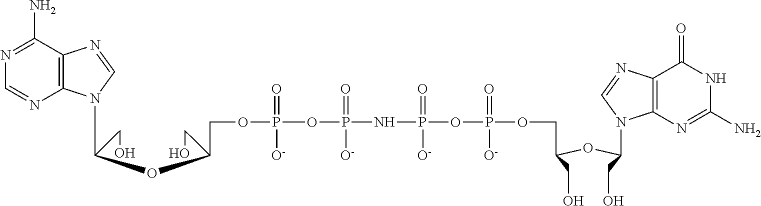 [SMILES: Nc1nc2c(ncn2[C@@H](CO)O[C@@H](CO)COP(=O)([O-])OP(=O)([O-])NP(=O)([O-])OP(=O)([O-])OC[C@H](CO)O[C@H](CO)n2cnc3c(N)ncnc32)c(=O)[nH]1]

In a further preferred aspect of the invention, the dinucleoside polyphosphate analogue is AppCH₂ppA.

As demonstrated in the Examples of the present application, such dinucleoside polyphosphate analogues as described above show a potent anti-epileptic effect.

Dinucleoside polyphosphates of general formula (I) and their preparation are disclosed in WO 2006/082397.

Mechanism

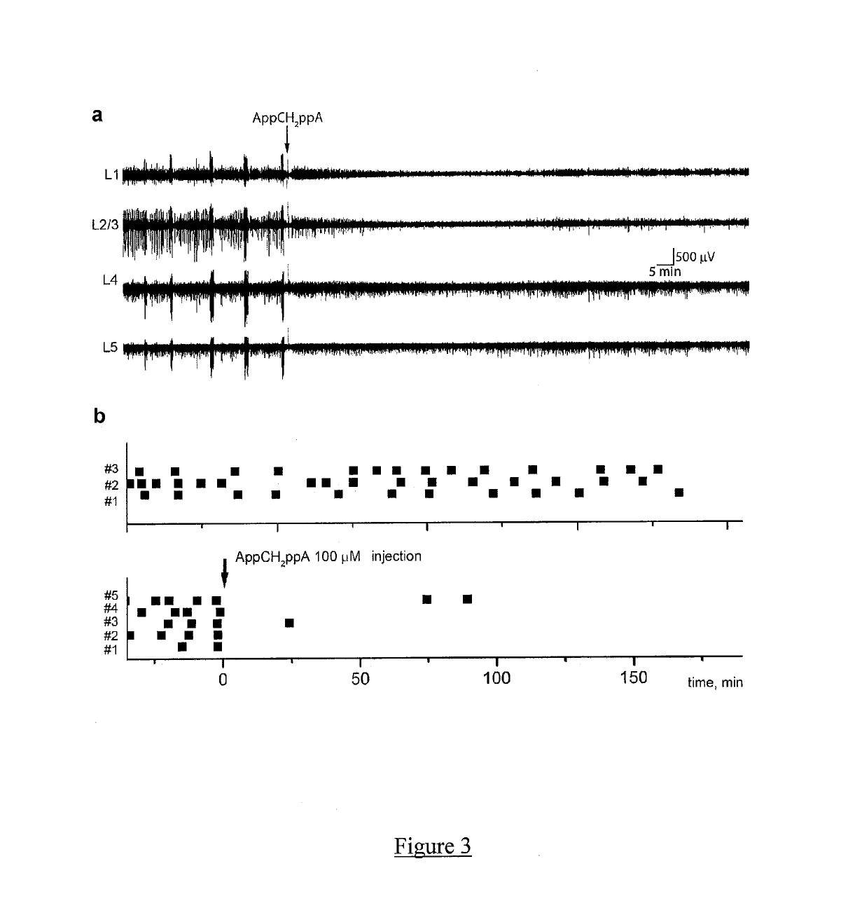

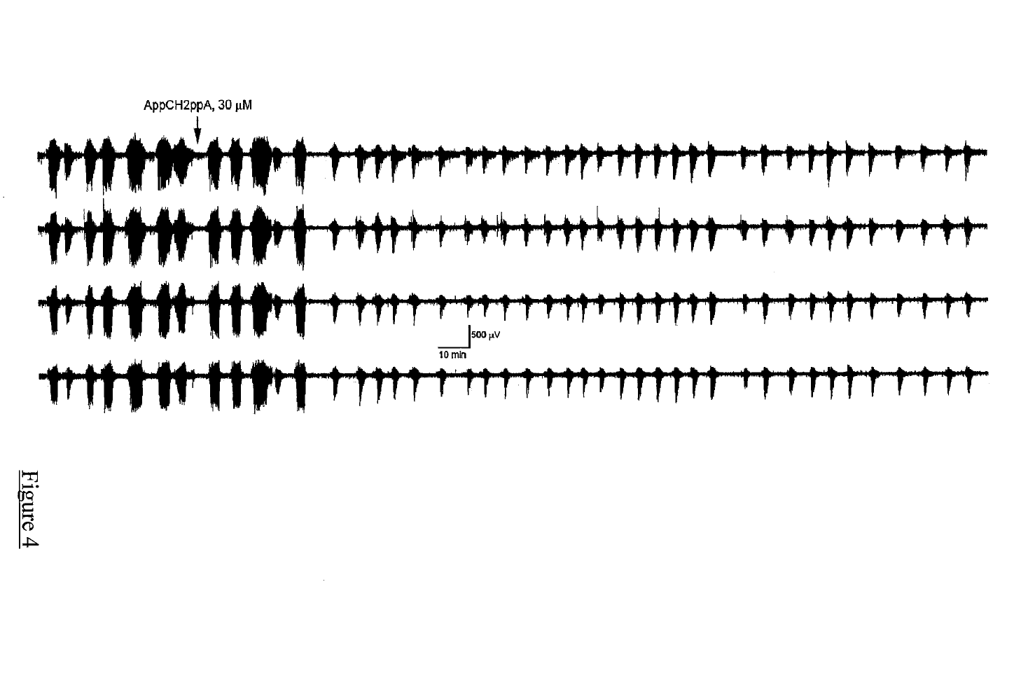

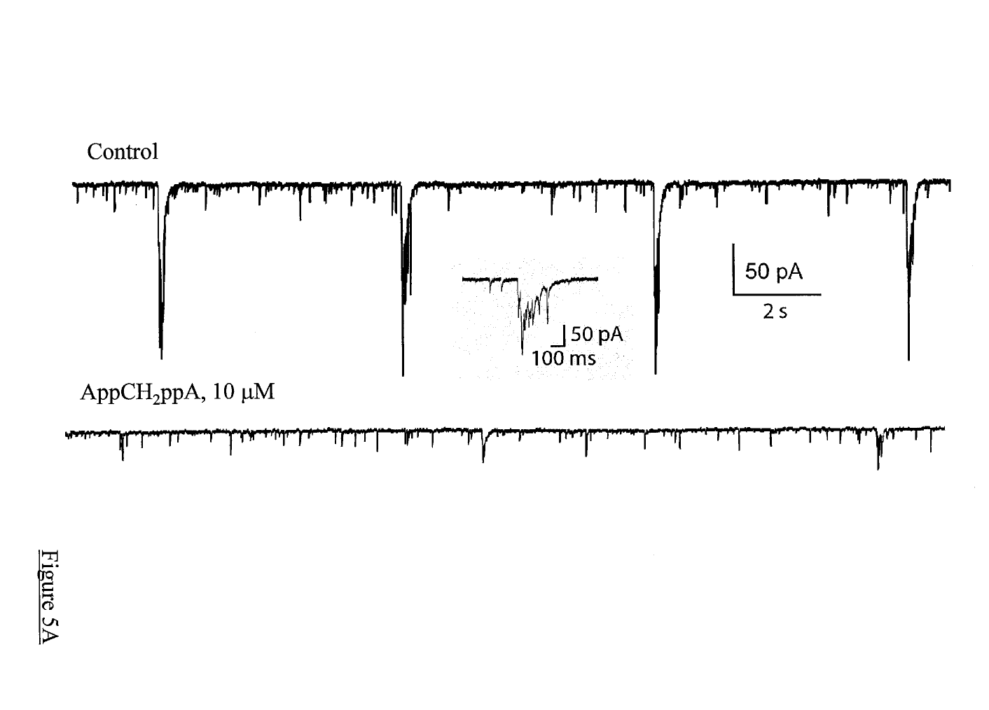

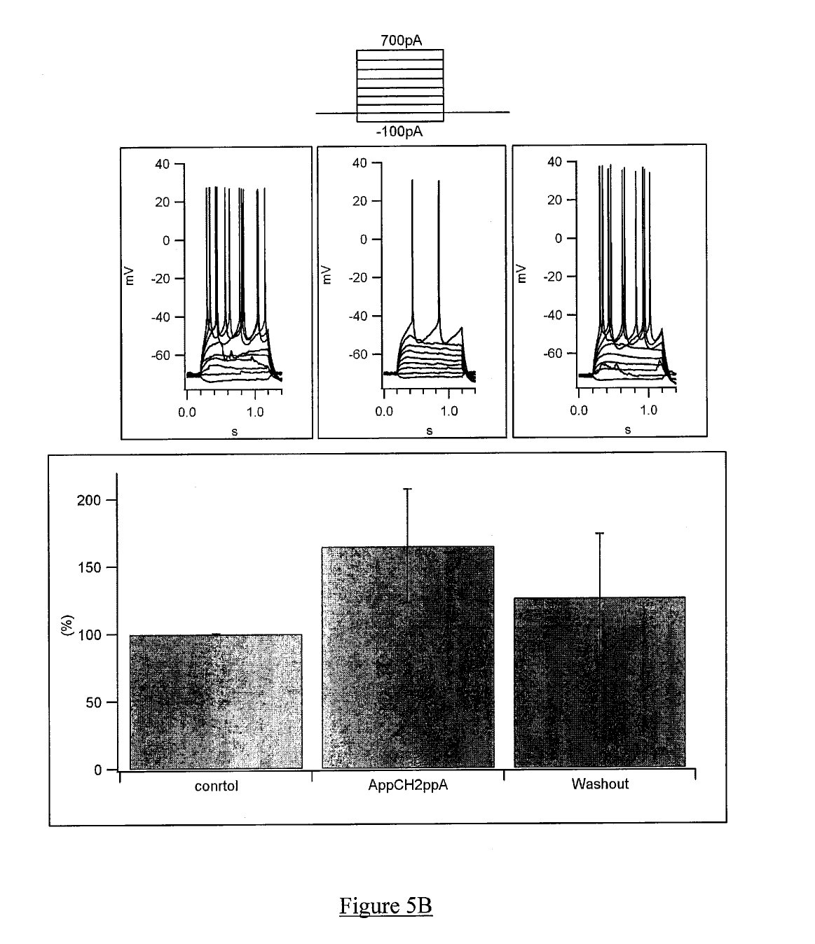

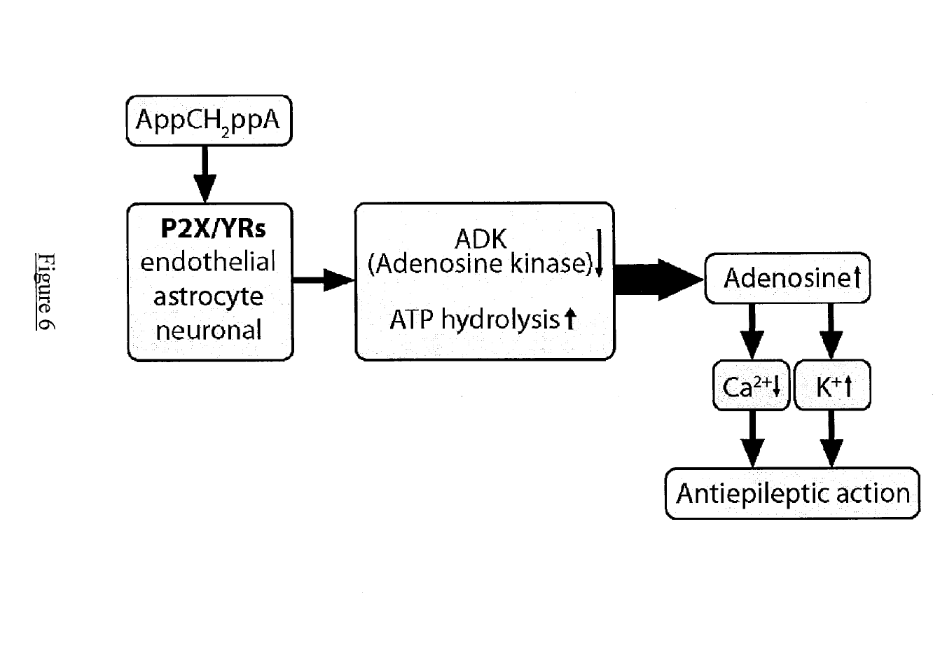

The present inventors have previously described how AppCH₂ppA has tissue protective properties in the brain by acting on an unknown P2X/Y receptor in order to elicit downstream production of adenosine. Adenosine was then seen to act on Al receptors causing neuroprotection (Melnik S, Wright M, Tanner J A, Tsintsadze T, Tsintsadze V, Miller A D, Lozovaya N (2006) Diadenosine polyphosphate analog controls postsynaptic excitation in CA3-CA1 synapses via a nitric oxide-dependent mechanism. J Pharmacol Exp Ther 318 (2):579-588. doi:10.1124/jpet.105.097642). Without wishing to be bound by theory, it is thought that the anti-epileptic effects now observed both ex vivo (FIG. 5) and in vivo (FIGS. 3 and 4) may be due to the endogeneous production of adenosine triggered by the administration of the dinucleoside polyphosphate analogue compounds. A proposed mechanism is set out in FIG. 6. It has previously been suggested that the (exogeneous) administration of adenosine could be a strategy for the treatment of epilepsy in human subjects (Boison D (2005) Adenosine and epilepsy: from therapeutic rationale to new therapeutic strategies. The Neuroscientist 11 (1):25-36. doi:10.1177/1073858404269112). However the present inventors have now found that the endogeneous generation of adenosine using the dinucleoside polyphosphate analogue compounds of the present invention surprisingly provides a highly potent anti-epileptic effect.

Thus in a preferred embodiment of the present invention, the dinucleoside polyphosphate analogues are for use in the treatment or prevention of epilepsy, such as juvenile epilepsy. In particular, the dinucleoside polyphosphate analogues may be for use in the treatment of pharmacoresistant epileptic syndromes, including Tuberous Sclerosis Complex (TSC). Thus in one preferred embodiment, the dinucleoside polyphosphate analogues are for use in the treatment or prevention of seizures associated with Tuberous Sclerosis Complex (TSC).

The present invention also relates to a method of treating or preventing epilepsy, comprising administering an effective amount of a dinucleoside polyphosphate analogue (as described herein) or a pharmaceutically acceptable salt thereof, and to use of a dinucleoside polyphosphate analogue (as described herein) or a pharmaceutically acceptable salt thereof, in the manufacture of a medicament for the treatment or prevention of epilepsy.

Dosages

The dinucleoside polyphosphate analogue of the present invention is preferably administered in an amount of about 10 to 500 nmol/kg, preferably from 12 to 75 nmol/kg, more preferably from 25 to 50 nmol/kg. Thus for example the compound may be administered in an amount of from 6 to 500 μg/kg, preferably 10 to 75 μg/kg, more preferably from 12 to 50 μg/kg.

Optimal dosages are 10-200, such as 10-100, nmol/kg.

Preferably, the dinucleoside polyphosphate analogue is one of the preferred analogues described above. In particular, the present invention relates to a dinucleoside polyphosphate analogue for use in the treatment of epilepsy, preferably wherein the dinucleoside polyphosphate analogue is chosen among the group consisting of: AppCH₂ppA, AppNHpppU, A_{diol}ppCH₂ppA_{diol}, A_{diol}ppNHppA_{diol}, AppCH₂ppG, AppNHppG, A_{diol}ppCH₂ppG_{diol} and A_{diol}ppNHppG_{diol}; more preferably wherein the dinucleoside polyphosphate analogue is AppCH₂ppA.

When used for the treatment of epilepsy, the compound chosen among the group consisting of: AppCH₂ppA, AppNHpppU, A_{diol}ppCH₂ppA_{diol}, A_{diol}ppNHppA_{diol}, AppCH₂ppG, AppNHppG, A_{diol}ppCH₂ppG_{diol} and A_{diol}ppNHppG_{diol} is preferably administered in association with a pharmaceutically acceptable vehicle. The dose of compound administered (to a subject in need of treatment) can be from about 10 to 100 nmol/kg, preferably from 12 to 75 nmol/kg, more preferably from 25 to 50 nmol/kg. Thus for example the compound may be administered in an amount of from 6 to 500 μg/kg, preferably 10 to 75 μg/kg, more preferably from 12 to 50 g/kg.

For example, for a typical human of about 70 kg, the amount of the compound administered may be between about 0.7 and about 35 μmol, more preferably between about 0.8 and about 5 μmol, and even more preferably between about 1 and about 3.5 μmol.

The dinucleoside polyphosphate analogues of the present invention may be administered in a variety of dosage forms. Thus, the dinucleoside polyphosphate analogues may be administered orally, for example as tablets, troches, lozenges, aqueous or oily suspensions, dispersible powders or granules. The dinucleoside polyphosphate analogues may also be administered parenterally, either subcutaneously, transdermally (by injection), intravenously, intramuscularly, intrasternally or by infusion techniques. The dinucleoside polyphosphate analogues may also be administered rectally, for example in the form of a suppository, or topically (for example using patches, microneedles or an iontophoretic transdermal delivery device). A physician will be able to determine the required route of administration for each particular patient. Preferably, the dinucleoside polyphosphate analogues are administered intravenously or by subcutaneous injection.

Compositions

Preferably, the composition is formulated for subcutaneous injection.

The formulation of the dinucleoside polyphosphate analogues will depend upon factors such as the nature of the exact agent, whether a pharmaceutical or veterinary use is intended, etc. An agent for use in the present invention may be formulated for simultaneous, separate or sequential use.

The dinucleoside polyphosphate analogues are typically formulated for administration in the present invention with a pharmaceutically acceptable excipient (such as a carrier or diluents). The pharmaceutical carrier or diluent may be, for example, an isotonic solution. For example, solid oral forms may contain, together with the active compound, diluents, e.g. lactose, dextrose, saccharose, cellulose, corn or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Formulations for oral administration may be formulated as controlled release formulations, for example they may be formulated for controlled release in the large bowel.

Solutions for intravenous administration or infusion may contain as carrier, for example, sterile water or preferably they may be in the form of sterile, aqueous, isotonic saline solutions.

The dinucleoside polyphosphate analogues of the present invention may also be administered in, or in combination with, a nanoparticle carrier, to improve delivery and/or targeting of the analogues. They may be delivered topically and/or transdermally, in a topical and/or transdermal formulation, e.g. in a transdermal patch or device.

Another possible mode of administration is intrathecally and/or to the brain (e.g. as a bolus).

The dose of the dinucleoside polyphosphate analogues may be determined according to various parameters, especially according to the substance used; the age, weight and condition of the patient to be treated; the route of administration; and the required regimen.

Again, a physician will be able to determine the required route of administration and dosage for any particular patient. A typical daily dose is from about 6 to 1000 μg per kg of body weight, according to the age, weight and conditions of the individual to be treated, the type and severity of the condition (e.g. of the eplilepsy) and the frequency and route of administration. Daily dosage levels may be, for example, from 6 to 500 μg/kg, preferably from about 10 to 100 μg/kg, more preferably from 12 to 75 μg/kg.

The dinucleoside polyphosphate analogues as described herein may be administered alone or in combination. They may also be administered in combination with another pharmacologically active agent, such as another agent for the treatment of epilepsy, for example carbamazepine, clorazepate, clonazepam, ethosuximide, felbamate, fosphenytoin, gabapentin, lacosamide, lamotrigine, levetiracetam, oxcarbazepine, phenobarbital, phenytoin, pregabalin, primidone, tiagabine, topiramate, valproate semisodium, valproic acid, and zonisamide. The combination of agents may be may be formulated for simultaneous, separate or sequential use.

Transdermal Delivery Devices

The compound can be administered in or by a device for transdermal delivery, so comprising a dinucleoside polyphosphate analogue or a pharmaceutically acceptable salt thereof. Such a physical delivery device can facilitate transport of the compound of interest into or across the skin barrier.

The device may be in the form of a patch containing the dinucleoside polyphosphate analogue and optionally a pharmaceutically acceptable excipient. The dinucleoside polyphosphate analogue may be dissolved, for example, in a gel and/or adhesive carrier on the patch.

Alternatively, the device (which may or may not be a patch) may comprise microneedles, for example in an array. Microneedles are typically no more than a micron in size: they may be able to penetrate the upper layer of the skin, for example without reaching nerves. The use of microneedles can thus facilitate transport of macromolecules across the skin barrier. Microneedles can be sharp and robust enough to easily penetrate the outer layer of skin. Due to their length can be such that they do not stimulate nerve cells deeper within the skin layers, the delivery of therapeutic agents can be pain-free. Furthermore, the use of microneedles can provide a slow release of the compounds to be delivered, since these are gradually released over time.

The device can be an iontophoretic (transdermal) delivery device (or patch) comprising a pharmaceutically acceptable salt of a dinucleoside polyphosphate analogue. Such a device can make use of iontophoresis, or electromotive drug administration (EMDA), to move or deliver the dinucleoside polyphosphate analogue (and any other compounds of interest) through or into the skin. Such a device enables efficient, non-invasive delivery of compounds of interest through the skin. It can thus cause the compound to flow diffusively (into or through the skin), for example driven by an electric field. The device may be portable and/or attachable to the skin or body, e.g. similar to a Zecuity™ patch machine (used for migraine but can comprise compounds of the invention).

Preferred salts of the dinucleoside polyphosphate analogue for use in an iontophoretic transdermal delivery device are as described above.

Nanoparticle(s)

The dinucleoside polyphosphate analogue or a pharmaceutically acceptable salt thereof may be combined with (e.g. linked to, inside, comprising, associated or formulated with or encapsulated within) a nanoparticle carrier, and a pharmaceutically acceptable excipient, or a (nano) particle comprising such an analogue (or salt).

Suitable exemplary nanoparticle carrier systems are lipid-based (or containing) nanoparticles, polymer-based (or containing) nanoparticles, inorganic nanoparticles and bioconjugates. The compound may be located in the core/on the or inside a lipid (bi)layer(s) which may be generally spherical. The particle may have multiple (e.g. concentric and/or spherical) layers as well, e.g. comprising lipids and/or polymers. The particle may be able to self-assemble. These are discussed in more detail below.

All publications and patent applications mentioned in this specification are indicative of the level of those skilled in the art to which this invention pertains. All publications and patent applications are herein incorporated by reference to the same extent as if each individual publication or patent application was specifically and individually to be incorporated by reference.

Although the foregoing invention has been described in some detail by way of illustration and example for purposes of understanding, it will be clear to those skilled in the art that certain changes and modifications may be practiced within the scope of the appended claims.

The following Examples illustrate the invention:

### EXAMPLES

### Ap₄a Analogue Synthesis

AppCH₂ppA was prepared using a development of the LysU-mediated biosynthetic process described previously (Melnik et al., 2006, WO 2006/0823297), with rigorous purification by HPLC (Wright et al., 2003, 2004 and 2006).

### In Vivo Recordings and Data Analysis

This study followed the Institut National de la Santé et de la Recherche Médicale guidelines for animal care. All experiments were performed on postnatal days P9-P20 of inbred C57Bl6 strain of both sexes of Tsc1^{mut/wt} (Tsc1^{+/-}) mice issued from breeding of C57Bl6 Tsc1^{wt} females and Tsc1^{mut/wt} males Tsc1^{mut/wt}.

Surgery was performed under isoflurane anesthesia. In brief, the skull of the animal was cleaned of skin and periosteum. The skull was covered by glue and dental cement except for a 4-9 mm² window above the somatosensory cortex from one or two hemispheres. Two plastic bars were fixed to the nasal and occipital bones of the pups head by dental cement. After surgery, animals were warmed, and left for an hour for recovery from anesthesia. During recordings, the head was fixed to the frame of the stereotaxic apparatus by attached bars; animals were surrounded by a cotton nest and heated via a thermal pad (36.6° C.-37.7° C.). A silver chloride reference electrode was placed in the cerebellum or visual cortex.

Electroencephalography (EEG) recordings were performed in non-anesthetized head-restrained Tsc1^{+/-} and control Tsc1^{wt} mice. 16-site linear silicon probe (100 μm separation distance between recording sites, Neuronexus Technologies, MI) was placed into the somatosensory cortex using the Paxinos and Franklin atlas (2001) at coordinates: AP=2-2.5 mm, L=2-3 mm; 1.2-1.5 mm depth, to trace the columnar activity at all layers and CA1 zone of the hippocampus. Signals were amplified (×100) and filtered at 3 kHz using a 16 channel amplifier (A-M systems, Inc), digitized at 10 kHz and saved to hard disk of PC using Axoscope software (Molecular Devices, Sunnyvale, Calif., USA). Recordings were analyzed off-line using Clampfit and MATLAB software. In 10 experiments, saline solution (200 μL) n=3 or AppCH₂ppA (30 or 100 μM) n=7 was injected intraperitoneally (i.p.). After the recordings, position of silicone probe was verified visually by DiI staining of the electrode in 100 μm coronal sections from fixed brain. We considered that multiunit activity occurred in epileptic discharges if they appeared in a group of multiple spikes whose amplitude exceeded at least twice the background activity within a period lasting for at least 20 s. The first and last spikes of each discharge were used to define its onset and termination, respectively. For each discharge amplitude was defined as the amplitude of the largest spike of the discharge. During EEG recordings animals were monitored visually to determine behavioral correlates of each electrographic epileptic discharge. For EEG data analysis raw data were preprocessed using a custom-developed suite of programs in the MATLAB analysis environment. The wide-band signal was downsampled to 1000 Hz and used for local field potential signal. Local field potentials were analyzed by the custom-written, MATLAB based programs. Approximate anatomical location of each recording site was estimated by physical depth within the brain and corresponding age-matched histological assessment of respective layers depth.

### Animal Slice Preparation

Wild type and Tsc1^{+/-} mice (P14-P16) were anaesthetized with ether and killed by decapitation in agreement with the European Directive 86/609/EEC requirements. The brain was rapidly removed and placed in an oxygenated ice-cold saline buffer. Transverse 300 μm-thick coronal slices were cut using a vibratome (Leica VT1000S; Leica Microsystems Inc., Deerfield, Ill.) in ice-cold protecting solution oxygenated with 95% O₂ and 5% of CO₂. Prior to recording, slices were incubated in an artificial cerebrospinal fluid (ACSF) solution containing (in mM): 125 NaCl, 3.5 KCl, 1 CaCl₂, 2 MgCl₂, 1.25 NaH₂PO₄, 26 NaHCO₃, and 10 glucose, equilibrated at pH 7.3 with 95% O₂ and 5% CO₂ at room temperature (22-25° C.) for at least 1 h to allow recovery.

### Electrophysiological Recordings from Brain Slices

Slices were transferred to the recording chamber and perfused with oxygenated recording ACSF at 3 ml/min. Neurons were visualized using infrared differential interference contrast (IR-DIC) microscopy. Whole-cell patch-clamp recordings were performed at room temperature by using either an EPC-9 amplifier and Patch Master software (HEKA Elektronik, Germany) or Multiclamp 700B amplifier (Molecular Devices, USA) and custom-made software based on IgorPro and filtered at 3-10 kHz. Patch pipettes were pulled from borosilicate glass capillaries (World Precision Instruments, Sarasota, USA) and had resistances of 4 to 6.5 MΩ when filled with the internal solution of the following composition (in mM): 130 K-gluconate, 10 Na-gluconate, 4 NaCl, 4 MgATP, 4 phosphocreatine, 10 HEPES, and 0.3 GTP (pH 7.3 with KOH). Biocytin (final concentration 0.3-0.5%) was added to the pipette solution to label the neurons from which recordings were obtained. The series resistance estimated from the amplitude of the initial capacitive transient in response to a 5-mV pulse was 8 to 24 MΩ. It was not compensated and was monitored during each experiment. Experiments were terminated if the series resistance changed by more than 15%. Spontaneous EPSCs were recorded for 30 min at −80 mV (the reversal potential for GABAergic currents) All recordings were made in normal ACSF (1 mM Mg²⁺) without the need for any pro-epileptic pharmacological drug. To minimize potential sampling bias, the pups from at least three deliveries for each condition were studied.

### Murine Hippocampal Slice Model of Epilepsy

Slices were prepared and used as described previously (Melnik S, Wright M, Tanner J A, Tsintsadze T, Tsintsadze V, Miller A D, Lozovaya N (2006) Diadenosine polyphosphate analog controls postsynaptic excitation in CA3-CA1 synapses via a nitric oxide-dependent mechanism. J Pharmacol Exp Ther 318 (2):579-588. doi:10.1124/jpet.105.097642).

### Example 1

### In Vivo Data—Antiepileptic Activity of AppCH₂ppA in Mouse Model of Tuberous Sclerosis

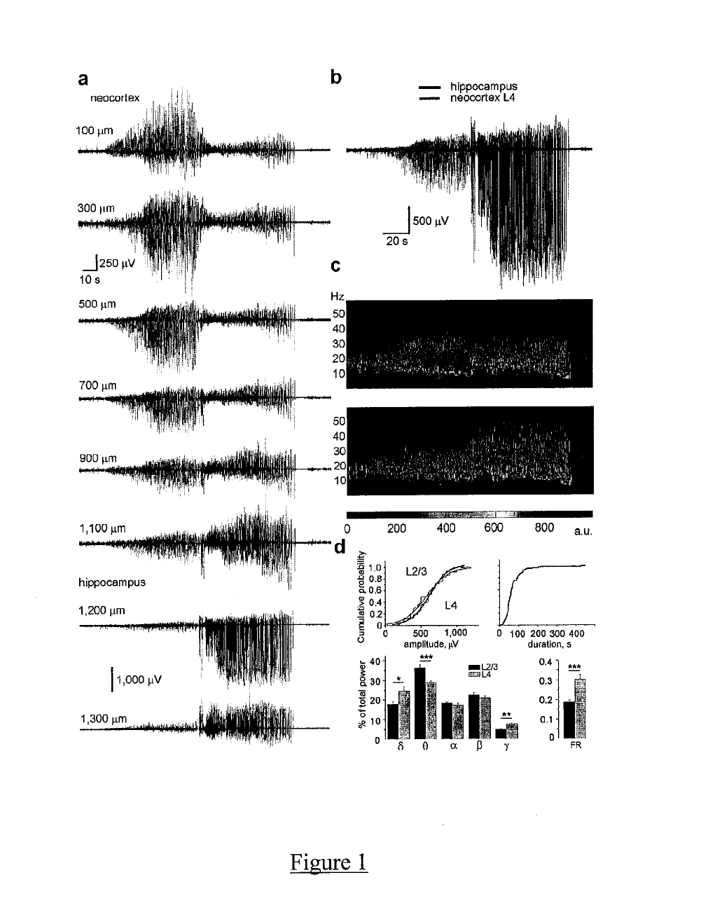

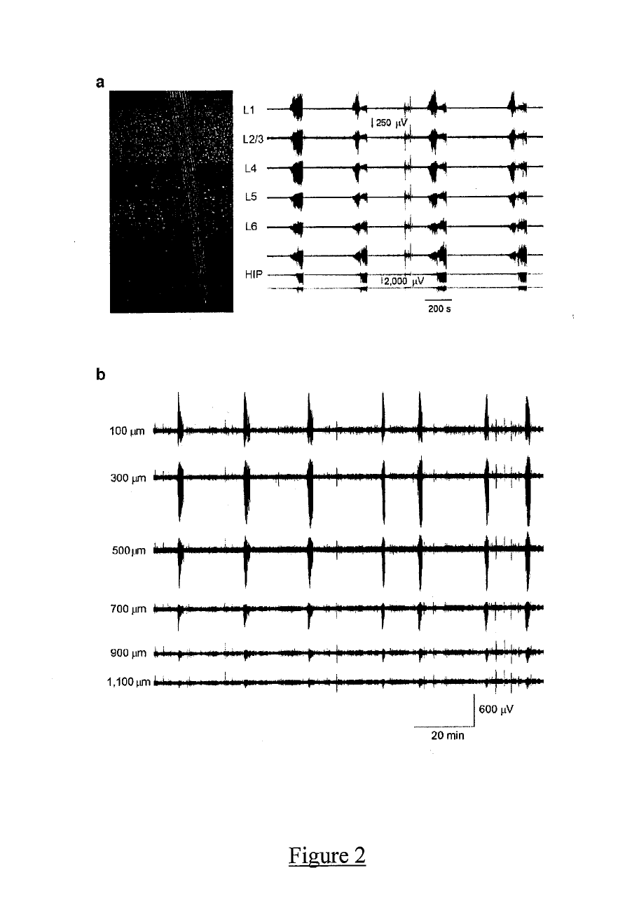

Tuberous Sclerosis Complex (TSC) is caused by dominant mutations in either TSC1 or TSC2 tumor suppressor genes, and is characterized by the presence of malformative brain lesions, namely cortical tubers that are thought to contribute towards the generation of pharmaco-resistant epilepsy. Tuberless heterozygote Tsc1^{+/-} mice exhibit recurrent, unprovoked seizures during early postnatal life (<P20). Seizures are generated intra-cortically in the granular layer of the neocortex. Details of the severe epilepsy generated in this model are shown (FIGS. 1 and 2).

When stable, synthetic dinucleoside polyphosphate analogue, AppCH₂ppA, was administered to Tsc1^{+/-} mice by intraperitoneal (i.p.) injection at a dose of 100 μM (in 200 μl) (20 nmol; 1000 nmol/kg or 0.84 mg/kg of animal body weight) then there was an essentially complete anti-epileptic effect (FIG. 3). When the experiment was repeated with a AppCH₂ppA dose of 30 μM (in 200 μl) (6 nmol; 300 nmol/kg or 0.25 mg/kg of animal body weight) (FIG. 4), then the effect on epilepsy was partial.

### Example 2

### Ex Vivo Data—Antiepileptic Activity of AppCH₂ppA in Mouse Cortical Slices

Wild type and Tsc1^{+/-} mice (P14-P16) were anaesthetized, their brains removed rapidly and placed in an oxygenated ice-cold saline buffer. Prior to recording, slices were incubated in an artificial cerebrospinal fluid (ACSF). The effects of AppCH₂ppA administration were monitored post slice administration ex vivo. Untreated slices were also studied for control comparisons (FIG. 5). The slice work demonstrates that AppCH₂ppA inhibits seizure like electrical impulses ex vivo on individual cortical neurons, as well as in the whole animal.

### Example 3

### Ex Vivo Data—Antiepileptic Activity of AppCH₂ppA in Mouse Hippocampal Slices

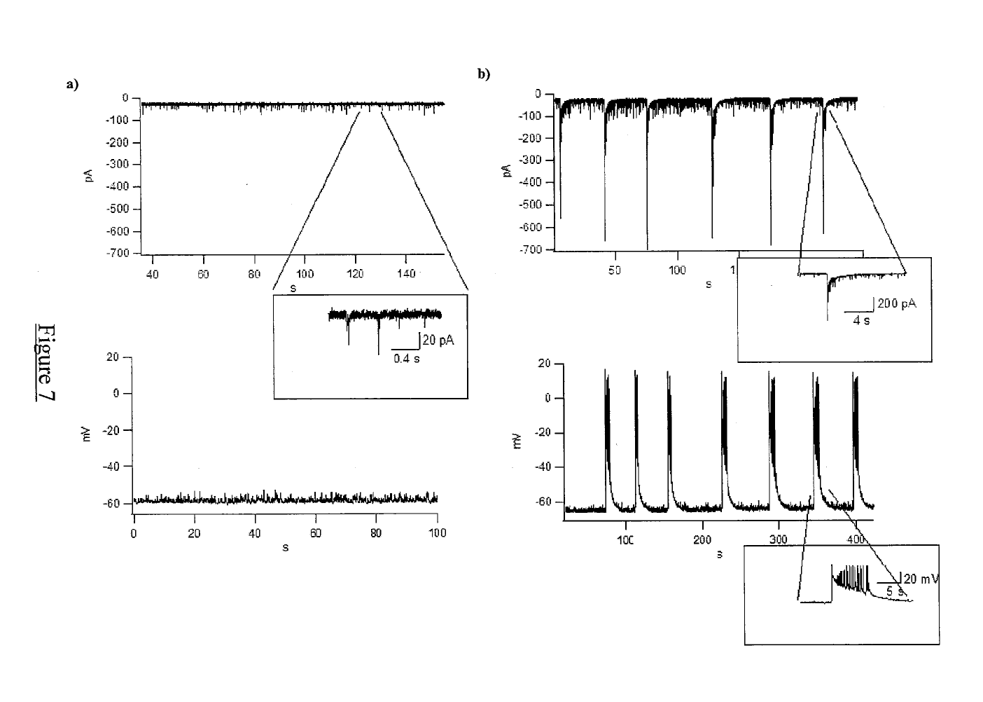

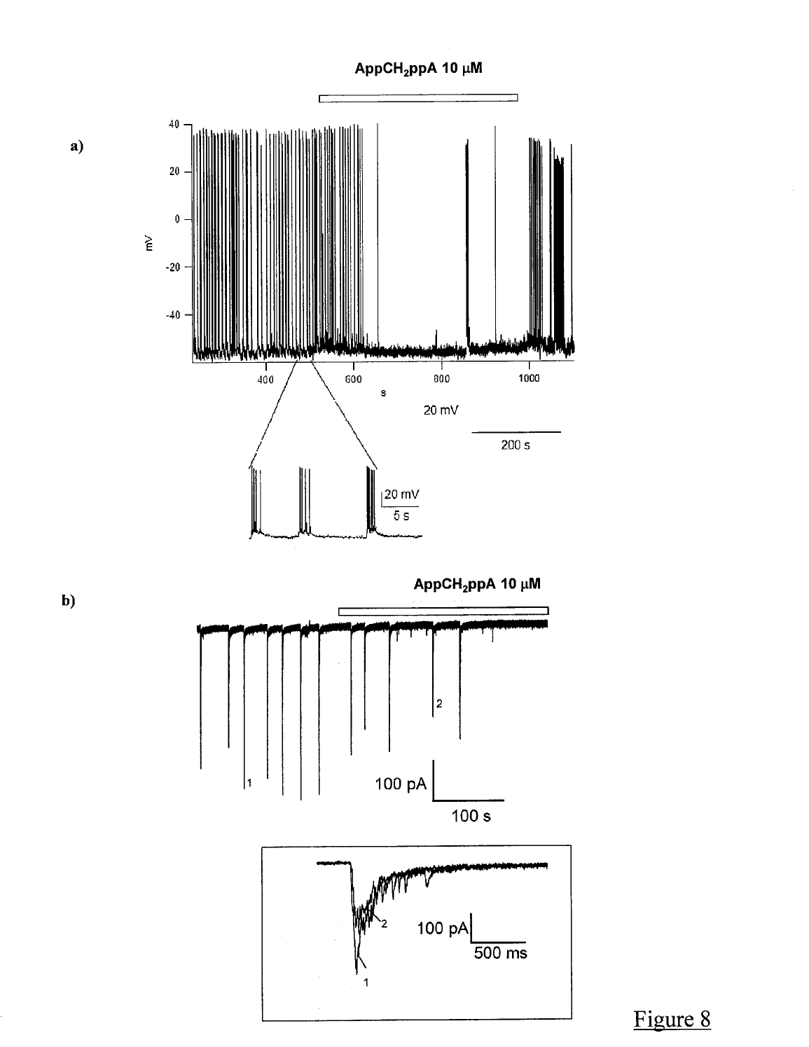

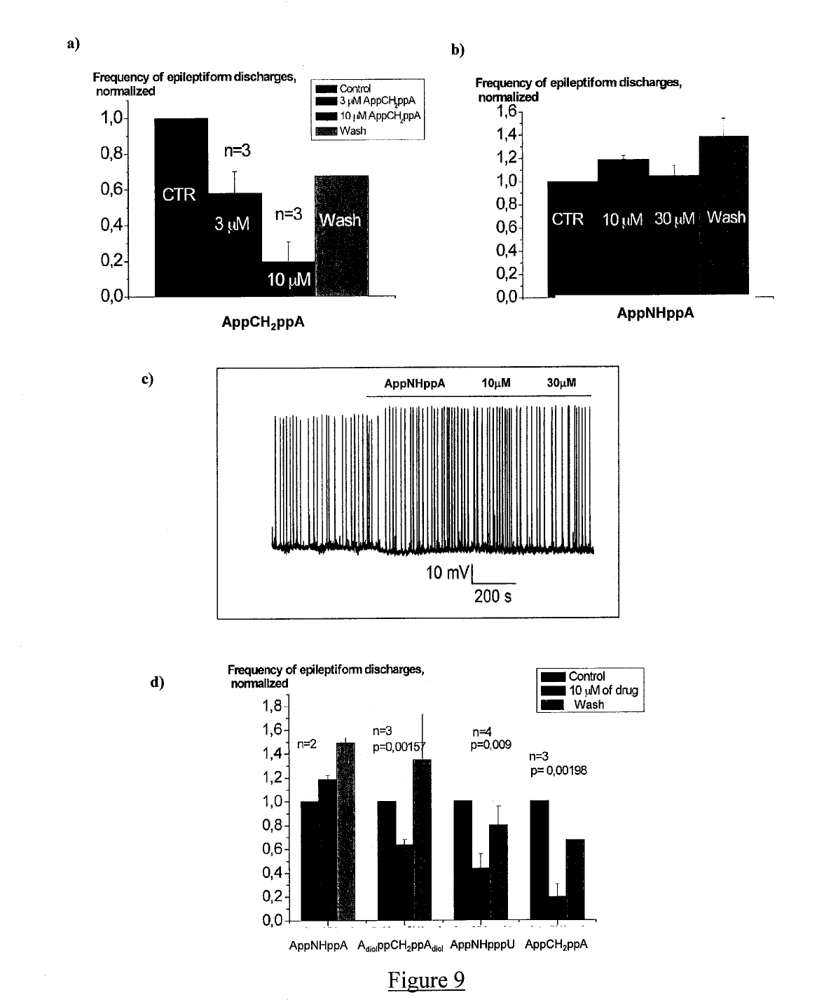

Slices were prepared as described previously (Melnik S, Wright M, Tanner J A, Tsintsadze T, Tsintsadze V, Miller A D, Lozovaya N (2006) Diadenosine polyphosphate analog controls postsynaptic excitation in CA3-CA1 synapses via a nitric oxide-dependent mechanism. J Pharmacol Exp Ther 318 (2):579-588. doi:10.1124/jpet.105.097642). Addition of picrotoxin (100 μM) and removal of Mg²⁺ in the perfusion solution induced spontaneous epileptiform events lasted for 5-10 s (FIG. 7). These events appeared initially at a low rate in the first few mins after the beginning of the picrotoxin perfusion, and gradually increased in rate, reaching a plateau frequency of approximately 6-8 events/5 min within 20-30 min. In the continued presence of picrotoxin and Mg²⁺ free extracellular solution, bursting at this rate continued for at least 2 h. The effects of AppCH₂ppA administration were monitored post slice administration ex vivo (FIGS. 8 and 9a). Importantly the stable, synthetic analogue AppNHppA was found completely inactive and other analogues of intermediate efficacy (FIGS. 9b-d).

All publications mentioned in the above specification are herein incorporated by reference. Various modifications and variations of the described methods and systems of the invention will be apparent to those skilled in the art without departing from the scope and the spirit of the invention. Although the invention has been described in connection with specific preferred embodiments, it should not be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A dinucleoside polyphosphate analogue, or a pharmaceutically acceptable salt thereof, for use as an anticonvulsant and/or seizure suppressant.

2. A dinucleoside polyphosphate analogue for use according to claim 1 wherein said dinucleotide polyphosphate analogue is a compound of formula (I): 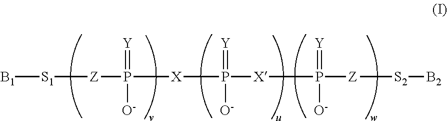
or a pharmaceutically acceptable salt thereof, wherein
X, X′ and Z are independently selected from 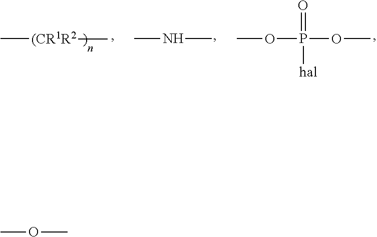
wherein R¹ and R² are independently selected from hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl, and n is selected from 1, 2, 3, 4, 5 and 6;
each Y is independently selected from ═S and =0;
B₁ and B₂ are independently selected from a 5- to 7-membered carbon-nitrogen heteroaryl group which may be unfused or fused to a further 5- to 7-membered carbon-nitrogen heteroaryl group
S₁ and S₂ are independently selected from a bond, C₁₋₆ alkylene, C₂₋₆ alkenylene, C₂₋₆ alkynylene and a moiety of formula (II): 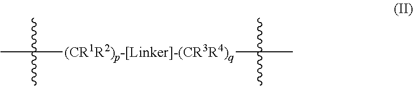
wherein
R¹, R², R³ and R⁴ independently represent hydrogen, halogen, hydroxyl, cyano or an unsubstituted group selected from C₁₋₃ haloalkyl, C₁₋₃ alkyl, C₁₋₃ aminoalkyl and C₁₋₄ hydroxyalkyl;
p and q independently represent 0, 1, 2 or 3, preferably 0, 1 or 2; and
[Linker] represents:
(i) —O—, —S—, —C═O— or —NH—;
(ii) C₁₋₄ alkylene, C₂₋₄ alkenylene or C₂₋₄ alkynylene, which may optionally contain or terminate in an ether (—O—), thioether (—S—), carbonyl (—C═O—) or amino (—NH—) link, and which are optionally substituted with one or more groups selected from hydrogen, hydroxyl, halogen, cyano, —NR⁵R⁶ or an unsubstituted group selected from C₁₋₄ alkyl, C₂₋₄ alkenyl, C₂₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; or
(iii) a 5 to 7 membered heterocyclyl, carbocyclyl or aryl group, which may be optionally substituted with one or more groups selected from hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl;
V is selected from 0, 1, 2, 3, 4 and 5;
U is selected from 0, 1, 2, 3, 4 and 5;
W is selected from 0, 1, 2, 3, 4 and 5; and
V plus U plus W is an integer from 2 to 7.

3-29. (canceled)
